(19) United States
(12) Patent Application Publication
(10) Pub. No.: US 2025/0171381 A1
(43) Pub. Date: May 29, 2025

(54) PROCESS FOR REGENERATING OLIGOMERIZATION CATALYST WITH PURGE

(71) Applicant: UOP LLC, Des Plaines, IL (US)

(72) Inventors: Suheil F. Abdo, Lincolnshire, IL (US); Manuela Serban, Northbrook, IL (US); Ashish Mathur, Gurgaon (IN)

(21) Appl. No.: 18/914,002

(22) Filed: Oct. 11, 2024

Related U.S. Application Data
(60) Provisional application No. 63/603,452, filed on Nov. 28, 2023.

(51) Int. Cl. C07C 2/12

(52) U.S. Cl. CPC C07C 2/12; C07C 2529/76

(57) ABSTRACT

A process for oligomerizing and oligomerizing olefins to distillate fuels which regenerates a first stage oligomerization catalyst bed and/or a second oligomerization catalyst bed in situ by contact with an oxygen gas at elevated temperature. Purge and pressurization of the oligomerization catalyst bed before regeneration and pressure maintenance after regeneration can be achieved with an inert gas provided by carbon dioxide generated from fermenting starches and sugars from biomass to make alcohol or from carbon burn in the regeneration process. The alcohol can also be dehydrated to olefins to provide the olefin charge stream for oligomerization. The oligomerization catalyst can be restored to full activity. A regeneration process can enable continuous operation.

### FIELD

The field is the conversion of olefins to distillate. The field may particularly relate to oligomerizing olefins and oligomerizing the oligomerized olefins to distillate fuels.

### BACKGROUND

An ethanol to jet fuel process is one of the routes that holds promise to minimize or eliminate net fossil-derived carbon combustion. The end product of this process is jet and diesel fuel produced out of bioethanol. The jet fuel produced in this manner is a sustainable aviation fuel intended to replace jet fuel produced out of conventional sources such as crude oil.

Bioethanol can be produced by fermentation of biological feedstock. Fermentation produces substantial carbon dioxide which must be managed. The bioethanol is then dehydrated to produce ethylene.

Ethylene can be dimerized and oligomerized into olefins such as C4, C6 and C8 olefins. Olefin oligomerization is a process that can oligomerize smaller olefins into larger olefins. More specifically, it can convert olefins into distillates including jet fuel and diesel range products. The olefinic oligomerized distillate can be hydrogenated for use as transportation fuel.

Jet fuel is one of the few petroleum fuels that cannot be replaced easily by electrical motor systems because a high energy output is required to power planes which cannot be supplied with electric motors. Large incentives are currently available for green jet fuel in certain regions to reduce the environmental impact of fossil-derived jet fuels. An efficient process is desired for producing distillate fuels from sustainable sources.

Improved integration of the fermentation process that converts starches to ethanol with dehydration of the ethanol to produce ethylene and subsequent oligomerization of the ethylene to produce sustainable aviation fuel (SAF) and other distillate fuels would be highly desirable from economic and sustainability perspectives. Oligomerization catalysts must be regenerated periodically to maintain catalyst activity.

### BRIEF SUMMARY

We have formulated a process for converting bioethanol to distillate fuels which regenerates oligomerization catalyst beds in situ by contact with an oxygen gas at elevated temperature. Carbon dioxide generated from fermentation of saccharides to ethanol or from regenerating oligomerization catalyst can be used to purge the oligomerization catalyst bed prior to coke burn and perhaps as a diluent for the oxygen gas. Ethanol from the process can be dehydrated to make olefins charged to the oligomerization unit.

### BRIEF DESCRIPTION OF THE DRAWINGS

FIG. 1 is a schematic drawing of a process and apparatus for making alcohol of the present disclosure.

FIG. 2 is a schematic drawing of a process and apparatus for dehydrating alcohol to olefins of the present disclosure.

FIG. 3 is a schematic drawing of an oligomerization section of a process and apparatus of the present disclosure.

FIG. 4 is a schematic drawing of a hydrogenation section of a process and apparatus of the present disclosure.

FIG. 5 is a schematic drawing of a regeneration process for the oligomerization reactor of a process and apparatus of the present disclosure.

FIG. 6 is a plot of conversion over time before and after regeneration.

FIG. 7 is a plot of selectivity over time before and after regeneration.

### DEFINITIONS

The term “communication” means that fluid flow is operatively permitted between enumerated components, which may be characterized as “fluid communication”.

The term “downstream communication” means that at least a portion of fluid flowing to the subject in downstream communication may operatively flow from the object with which it fluidly communicates.

The term “upstream communication” means that at least a portion of the fluid flowing from the subject in upstream communication may operatively flow to the object with which it fluidly communicates.

The term “direct communication” means that fluid flow from the upstream component enters the downstream component without passing through any other intervening vessel.

The term “indirect communication” means that fluid flow from the upstream component enters the downstream component after passing through an intervening vessel.

The term “bypass” means that the object is out of downstream communication with a bypassing subject at least to the extent of bypassing.

As used herein, the term “predominant” or “predominate” means greater than 50%, suitably greater than 75% and preferably greater than 90%.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the vapor outlet of the column. The bottom temperature is the liquid bottom outlet temperature. Overhead lines and bottoms lines refer to the net lines from the column downstream of any reflux or reboil to the column. Stripper columns may omit a reboiler at a bottom of the column and instead provide heating requirements and separation impetus from a fluidized inert media such as steam. Stripping columns typically feed a top tray and take main product from the bottom.

As used herein, the term “separator” means a vessel which has an inlet and at least an overhead vapor outlet and a bottoms liquid outlet and may also have an aqueous stream outlet from a boot. A flash drum is a type of separator which may be in downstream communication with a separator that may be operated at higher pressure. As used herein, the term “boiling point temperature” means atmospheric equivalent boiling point (AEBP) as calculated from the observed boiling temperature and the distillation pressure, as calculated using the equations furnished in ASTM D1160 appendix A7 entitled “Practice for Converting Observed Vapor Temperatures to Atmospheric Equivalent Temperatures”.

As used herein, the term “True Boiling Point” (TBP) means a test method for determining the boiling point of a material which corresponds to ASTM D-2892 for the production of a liquefied gas, distillate fractions, and residuum of standardized quality on which analytical data can be obtained, and the determination of yields of the above fractions by both mass and volume from which a graph of temperature versus mass % distilled is produced using fifteen theoretical plates in a column with a 5:1 reflux ratio.

As used herein, the term “T5”, “T90” or “T95” means the temperature at which 5 mass percent, 90 mass percent or 95 mass percent, as the case may be, respectively, of the sample boils using ASTM D-86 or TBP.

As used herein, the term “initial boiling point” (IBP) means the temperature at which the sample begins to boil using ASTM D-7169, ASTM D-86 or TBP, as the case may be.

As used herein, the term “end point” (EP) means the temperature at which the sample has all boiled off using ASTM D-7169, ASTM D-86 or TBP, as the case may be.

As used herein, the term “diesel” means hydrocarbons boiling in the range of an IBP between about 125° C. (257° F.) and about 175° C. (347° F.) or a T5 between about 150° C. (302° F.) and about 200° C. (392° F.) and the “diesel cut point” comprising a T95 between about 343° C. (650° F.) and about 399° C. (750° F.) using the TBP distillation method or a T90 between 280° C. (536° F.) and about 340° C. (644° F.) using ASTM D-86. The term “green diesel” means diesel comprising hydrocarbons not sourced from fossil fuels.

As used herein, the term “jet fuel” means hydrocarbons boiling in the range of a T10 between about 190° C. (374° F.) and about 215° C. (419° F.) and an end point of between about 290° C. (554° F.) and about 310° C. (590° F.). The term “green jet fuel” means jet fuel comprising hydrocarbons not sourced from fossil fuels.

### DETAILED DESCRIPTION

The process disclosed involves using byproduct carbon dioxide produced during ethanol production by fermentation of biomass, or from the oligomerization catalyst regeneration to purge hydrocarbons from the oligomerization catalyst to prepare the catalyst for regeneration. The process utilizes a zeolitic catalyst for ethylene oligomerization in a first stage and a metal catalyst for olefins oligomerization in a second stage.

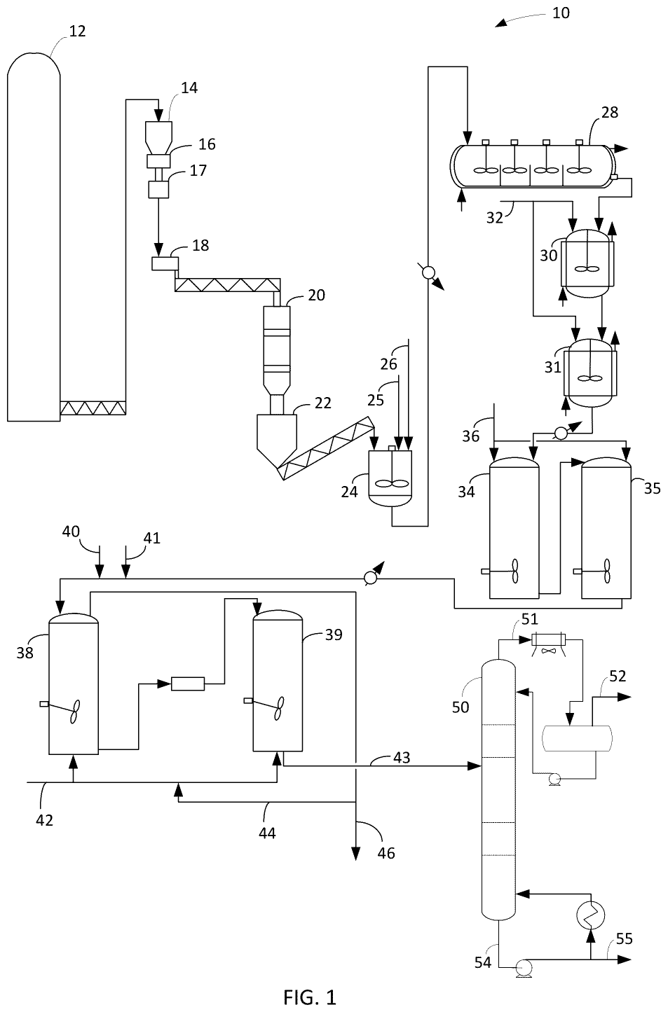

The process for making ethanol is illustrated in FIG. 1. Any renewable material that can provide saccharides will do. In this case, corn, the most prevalent renewable for making alcohol, is exemplified. In a typical process for making alcohol 10, shelled corn is delivered with debris removed and stored in a storage silo 12. Corn is conveyed from the silo to a weigh tank 14 and a series of separators 16 and 17. The separated corn kernels are transported to a dry destoner 18 to remove grit and stones and conveyed to a hammer mill 20. In the hammer mill corn kernels are ground to flour and collected in a flour bin 22.

The corn flour is conveyed from the bin 22 to a slurry tank 24 in which it is mixed with an enzyme such as alpha amylase from line 25 and aqueous lime from line 26. Alpha-amylase is an enzyme that catalyzes the hydrolysis of α-bonds of large, α-linked polysaccharides, such as starch and glycogen, yielding shorter chains thereof, dextrins, and maltose. The lime is added usually in the form of calcium hydroxide to disrupt the lignocellulosic matrix to make the substrate more accessible to the enzymes. The slurried mixture is conveyed after a short residence time such as 3 to 10 minutes and heated to about 70 to about 90° C. en route to a liquefaction tank 28.

The liquefaction tank 28 is heated by a steam jacket to maintain temperature of about 70 to about 90° C. Residence time is optimized to reduce formation of dextrin units which are not fermentable in yeast. The multi-stirred liquefaction tank retains the slurry from about 45 to about 75 minutes. The enzyme breaks the starch into soluble simpler starches, glucose and dextrose in a mash.

The mash is conveyed to one or more cooking kettles 30, 31 where heating continues by a steam jacket at a temperature of about 100 to about 120° C. while continually stirring. Sulfuric acid from line 32 may be added to the cooking kettles 30, 31 to break up and loosen any polymeric material such as lignin and cellulose. Residence time in the cooking kettles 30, 31 may be for about 10 to about 20 minutes to mitigate by-product formation of methanol and fusel oils. The cooked mash is cooled to between about 50 and about 70° C. and conveyed to the saccharification tanks 34, 35.

Enzyme such as glucoamylase is added to the saccharification tanks from line 36 to effect saccharification of the cooked mash at the reduced temperature under stirring to produce dextrin. Residence time in the saccharification tanks 34, 35 is about 1.5 to about 2.5 hours. Saccharified broth is cooled to about 30 to about 50° C. and conveyed to fermenters 38, 39.

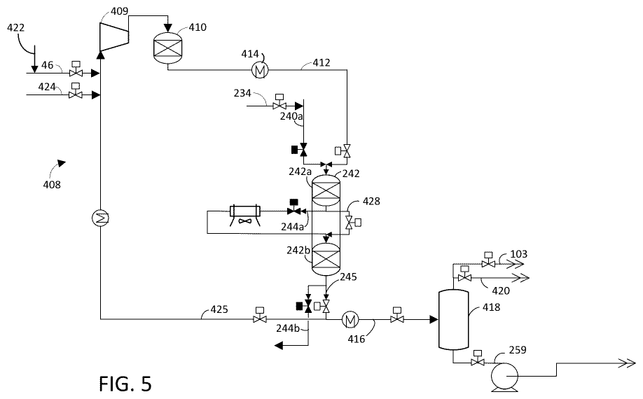

Nutrients in line 40 and anti-foaming agent in line 41 are also added to the fermenter 38. Air in line 42 is added to the bottom of the fermenters 38, 39 to promote ethanol production. Carbon dioxide from line 44 may also be added to the fermenters 38, 39 to promote further agitation in the fermenters. Carbon dioxide is also generated in the fermenters 38, 39. Some of the carbon dioxide is recycled in line 44 to the fermenters while surplus carbon dioxide is taken in line 46. The surplus carbon dioxide can be used for regeneration of oligomerization catalyst in oligomerization reactors 242, 252 in FIG. 5.

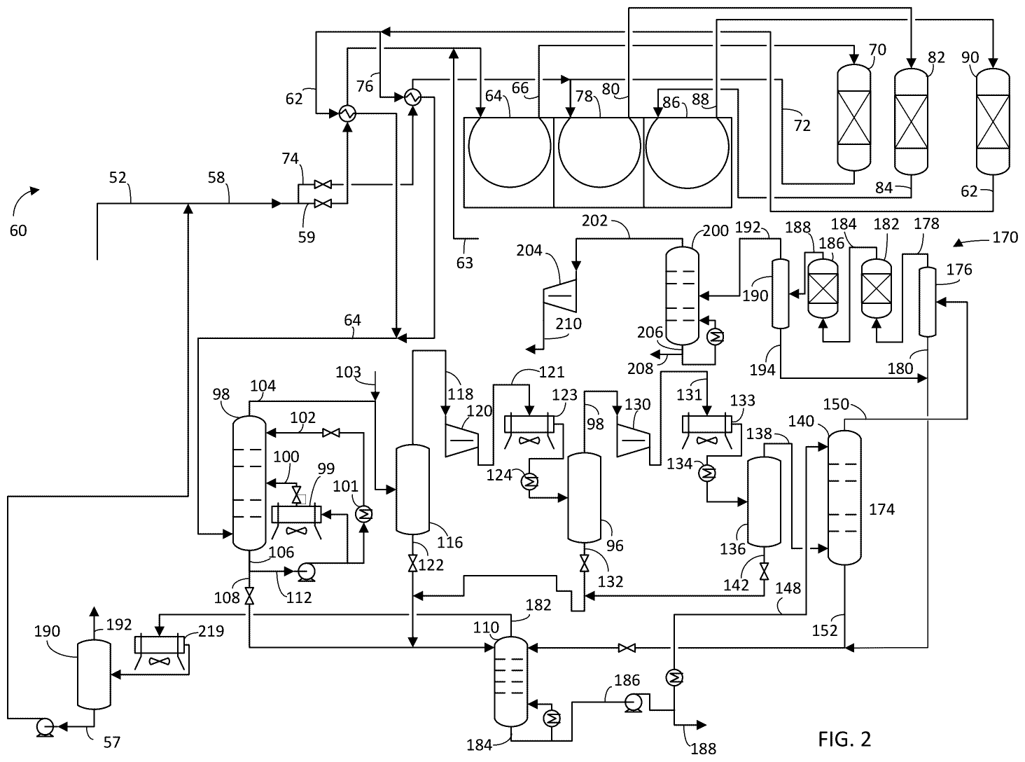

A beer alcohol stream in line 43 from the fermenters 38, 39 may be fed to a fractionation column 50 to concentrate the alcohol in the overhead line 51. The fractionated overhead stream may be condensed with a net vaporous alcohol stream provided from the receiver in a receiver overhead line 52 for charge to the dehydration unit 60 in FIG. 2. The condensed overhead stream may be refluxed to the column. Alternatively, the overhead stream in line 51 may be completely condensed with a portion of the condensed stream taken to a rectifier column to further concentrate the alcohol stream before it is taken to the dehydration unit 60. A bottoms stream in line 54 has a portion reboiled and returned to the column while a net bottoms stream comprising solids in non-volatile liquids is taken in line 55. The fractionation column 50 may be operated at around atmospheric pressure with an overhead temperature of about 60° C. (140° F.) to about 90° C. (194° F.) and a bottom temperature of about 90° C. (194° F.) to about 110° C. (230° F.). The rectifier column is run at

The dehydration unit 60 is shown in FIG. 2. The vaporous alcohol stream in line 52 is provided from the fractionation column 50. An ethanol recycle stream in line 57 may be combined with the net gaseous alcohol stream in line 52 to provide a charge gaseous ethanol stream in line 58. The charge gaseous ethanol stream in line 58 is split into two charge gaseous ethanol streams. A first charge gaseous ethanol stream in line 59 is heat exchanged with a first dehydrated exchange stream in line 62, mixed with steam in line 63 and fed to a first charge heater 64. The first charge heater 64 may be a fired heater and may heat the first charge gaseous ethanol stream to about 400° C. to about 550° C. Less heater duty is required to heat the first charge gaseous ethanol stream to reaction temperature required because the gaseous ethanol does not require the enthalpy of vaporization to enter into the vapor phase required for the dehydration reaction. A resulting first heated charge gaseous ethanol stream in line 66 is charged to a first dehydration reactor 70. In the first dehydration reactor 70, gaseous ethanol is converted to ethylene and water over a dehydration catalyst at a pressure of about 455 kPa (gauge) 65 psig to about 630 kPa (gauge) (90 psig). A first dehydrated stream is discharged from the first dehydration reactor 70 in line 72.

A second charge gaseous ethanol stream in line 74 is heat exchanged with a second dehydrated exchange stream in line 76, mixed with the first dehydrated stream in line 72 and fed to a second charge heater 78. The second charge heater 78 may be a fired heater and may heat the second charge stream to about 400° C. to about 550° C. Less heater duty is required to heat the second charge gaseous ethanol stream to reaction temperature required because the gaseous ethanol does not require the enthalpy of vaporization to enter into the vapor phase required for the dehydration reaction. A resulting second heated charge stream in line 80 is charged to a second dehydration reactor 82. In the second dehydration reactor 82, ethanol feed is converted to ethylene and water over a dehydration catalyst at a pressure of about 420 kPa (gauge) 60 psig to about 700 kPa (gauge) (100 psig). A second dehydrated stream is discharged from the second dehydration reactor 82 in line 84.

The second dehydrated stream in line 84 is fed to an interheater 86. The interheater 86 may be a fired heater and may heat the second dehydrated stream to about 400° C. to about 550° C. A resulting third heated charge stream in line 88 is charged to a third dehydration reactor 90. In the third dehydration reactor 90, residual gaseous ethanol feed is converted to ethylene and water over a dehydration catalyst at a pressure of about 420 kPa (gauge) 60 psig to about 700 kPa (gauge) (100 psig). A third dehydrated stream is discharged from the third dehydration reactor 90 in line 92.

The dehydration catalyst may be an alumina-based catalyst or based other supports which provide the desired level of acidity and surface area.

The third dehydrated stream is split between the first dehydrated exchange stream in line 62 and the second dehydrated exchange stream in line 76. The first dehydrated exchange stream in line 62 is heat exchanged with the first charge gaseous ethanol stream in line 60, and the second dehydrated exchange stream in line 76 is heat exchanged with the second charge gaseous ethanol stream in line 74 and the cooled dehydrated streams are recombined in line 64.

The recombined, cooled dehydrated stream in line 64 is fed to a quench tower 98 in which the cooled dehydrated stream is quenched by direct contact with water from a first cooled water stream in line 100 and a second cooled water stream in line 102. A quenched ethylene stream exits in a quench overhead line 104 and a bottoms water stream exits the tower bottoms in line 106. The bottoms water stream is split between a drain stream in line 108 which may be transported to a waste-water stripper column 110 through a valve thereon and a quench recycle stream in line 112. A first portion of the quench recycle stream is air cooled in a product condenser 99 and recycled as the first, lower cooled water stream in line 100 through a valve thereon, and a second portion of the quench recycle stream is heat exchanged in a trim condenser 101 and recycled to the quench tower 98 as the second, higher cooled water stream in line 102. The quench tower 98 may be operated with a bottom temperature of about 37° C. (100° F.) to about 104° C. (220° F.) and a pressure of about 280 kPa (gauge) (40 psig) to about 490 kPa (gauge) (70 psig) in the overhead.

The quenched ethylene stream in line 104 is fed to a first stage suction drum 116. Depressurized oligomerization reactor vapors in line 103 from a first stage oligomerization reactor 242 or a second stage oligomerization reactor 252 may also be added to the quenched ethylene stream and fed to the first stage suction drum 116. In the first stage suction drum ethylene exits in the overhead line 118 to a first stage compressor 120 while residual water exits the bottom of the drum in line 112 through a control valve thereon and is transported to the waste-water stripper column 110 perhaps via line 108. The first stage compressor 120 compresses the ethylene stream to a first pressure of about 350 kPa (gauge) (50 psig) to about 1225 kPa (gauge) (175 psig) and the discharge in line 121 is cooled in a first stage discharge cooler 123 and a first stage trim cooler 124.

The cooled, compressed ethylene stream from the first stage trim cooler 124 is fed to a first stage discharge drum 126. From the first stage discharge drum 126 ethylene exits in an overhead line 128 to a second stage compressor 130 while residual water exits a bottom of the drum in line 132 through a control valve thereon and is transported to the waste-water stripper column 110 perhaps via lines 122 and 108. The second stage compressor compresses the ethylene stream to a second pressure of about 455 kPa (gauge) (165 psig) to about 3220 kPa (gauge) (460 psig) and the discharge in line 131 is cooled in a second stage discharge cooler 133 and a second stage trim cooler 134.

The twice cooled, compressed ethylene stream from the second stage trim cooler 134 is fed to a second stage discharge drum 136. From the second stage discharge drum 136 ethylene exits in an overhead line 138 and is transported to a water wash tower 140 while a residual water stream exits the bottom of the drum in line 142 through a control valve thereon and is transported to the waste-water stripper column 110 perhaps via lines 132, 122 and 108.

In the water wash tower 140, the twice cooled, compressed ethylene stream is counter-currently washed with cooled, treated water in line 148 from the waste-water stripper column 110 to absorb additional oxygenates to produce a washed ethylene stream exiting in an overhead line 150 and a wash water stream in a bottoms line 152. The washed ethylene stream in the overhead line 150 is transported to a product drier section 170. The wash water stream in line 152 is transported back to the waste-water stripper column 110 through a valve thereon. The water wash tower 140 may be operated with a bottom temperature of about 16° C. (60° F.) to about 82° C. (150° F.) and a pressure of about 2800 kPa (gauge) (400 psig) to about 3500 kPa (gauge) (500 psig) in the overhead. The washed vaporous ethylene stream exiting the overhead of the water wash tower 140 in the overhead line 150 is fed to the product drier section 170.

In the product drier section 170, the washed ethylene stream in line 150 is fed to a first drier inlet knock-out drum 176 to remove residual water and provide a drier inlet stream in line 178 and a knock-out water stream in the bottoms line 180 which is fed to the waste-water stripper column 110 perhaps via line 152. The drier inlet stream is fed to a first product drier 182 in line 178. The first product drier 182 comprises an adsorbent for adsorbing the water from ethylene in the drier inlet stream in line 178 to provide a dried ethylene stream. The adsorbent may be a molecular sieve material with pore diameters of 2-4 A. The first product drier 182 may operate in up flow mode. The product drier section 170 may include a second product drier 186 that operates as the first product drier 182. The second product drier 186 may receive effluent from the first product drier in line 184. The two product driers may be operated in series but are preferably arranged in a lead-lag operation to facilitate regeneration during continuous operation. The second product drier 186 comprises an adsorbent for adsorbing the water from ethylene like in the first product drier 182. A dried ethylene stream exits the product drier section 170 in a dried ethylene stream in line 188. The product drier section 170 may be operated at a temperature of about 32° C. (90° F.) to about 49° C. (120° F.) and a pressure of about 2.8 MPa (gauge) (400 psig) to about 3.1 MPa (gauge) 450 psig).

The dried ethylene stream in line 188 is fed to a drier outlet knock-out drum 190 to remove residual water and provide a drier outlet stream in line 192 and a second knock-out water stream in a bottoms line 194 which is fed to the waste-water stripper column 110 perhaps via lines 180 and 152.

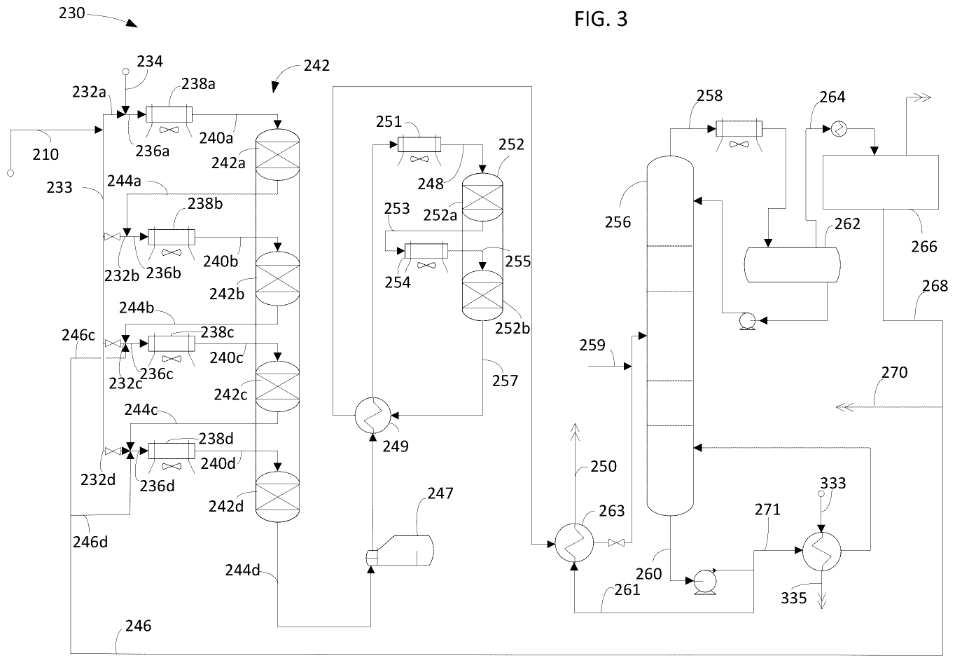

The drier outlet stream in line 192 may be fed to a heavy oxygenates removal column 200 to separate an overhead stream comprising predominantly ethylene but perhaps higher olefins from heavy ketones and diethyl ether. The olefins are produced in an overhead line 202 and fed to a third stage compressor 204 and a bottoms heavy oxygenate stream is produced in a bottoms line 206. A heavy oxygenate purge stream may be taken in line 208 to heavy oxygenate treatment while a reboil portion is reboiled and fed back to the column 200. A compressed ethylene stream at a pressure of about 2800 kPa (gauge) (400 psig) to about 7000 kPa (gauge) (1000 psig) in a compressor discharge line 210 may be provided to an oligomerization section 230 of FIG. 3. The heavy oxygenate removal column 200 may be operated with a bottom temperature of about −29° C. (−20° F.) to about 121° C. (250° F.) and a pressure of about 2.4 MPa (gauge) (350 psig) to about 3.1 MPa (gauge) (450 psig) in the overhead.

Water streams comprising oxygenates and volatiles in lines 122, 132, 142, 152, 180 and 194 may be fed to the waste-water stripper column 110 in which volatiles and oxygenates are boiled off to provide an overhead volatile stream in line 182 and a stripped water stream in line 184. A portion of the stripped water stream can be reboiled and fed back to the column to provide necessary heat. A treated water stream in line 186 may be pumped to water outlets which includes other water outlets in line 188 and the cooled, treated water stream in line 148 to the water wash tower 140. The waste-water stripper column 110 may be operated with a bottom temperature of about 93° C. (200° F.) to about 121° C. (250° F.) and a pressure of about 35 kPa (gauge) (5 psig) to about 138 kPa (gauge) (20 psig) in the overhead.

The overhead volatile stream in line 182 may be cooled in an air cooler 219 and fed to an off-gas knock out drum 220. An overhead stream from the knock-out drum 220 in line 222 may be sent to flare while an ethanol recycle stream is pumped in recycle line 57 to supplement the vaporous alcohol stream in the net overhead 52 to provide the charge gaseous ethanol stream in line 58.

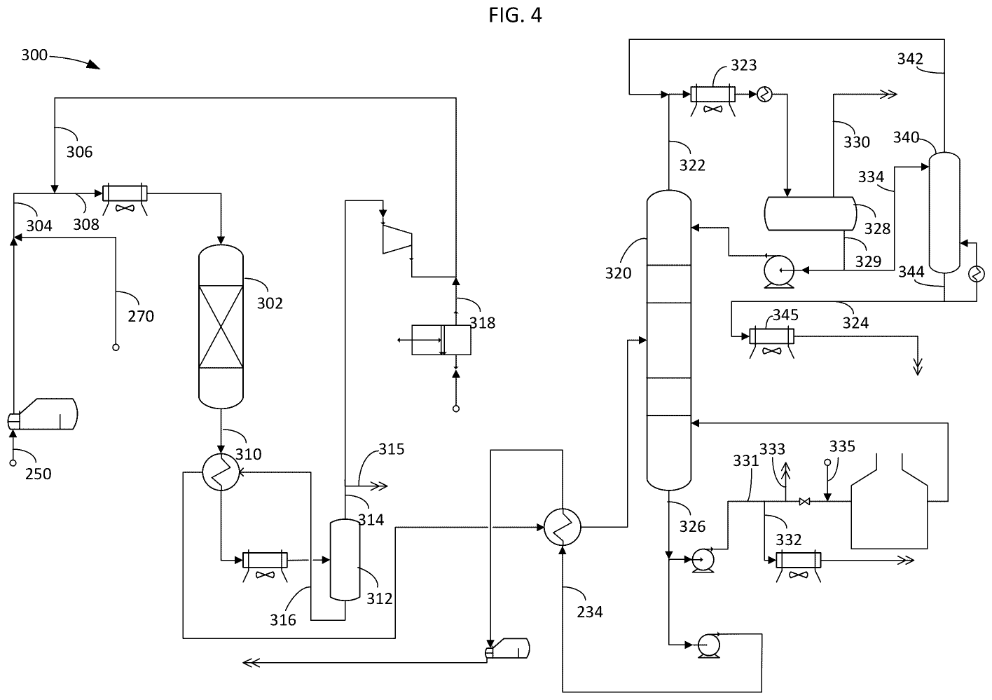

The process and apparatus may include an oligomerization section 230 in FIG. 3 and a hydrogenation section 300 in FIG. 4.

Turning first to the oligomerization section 230 of FIG. 3, a compressed olefin stream in line 210 from FIG. 2 is provided to the oligomerization section 230. The olefin stream may comprise substantial ethylene. The charge olefin stream may predominantly comprise ethylene. In an aspect, the olefin stream may comprise at least 95 mol % ethylene. The olefin stream in line 210 may be styled an ethylene stream. The olefin stream may be provided by the dehydration of ethanol or provided from a MTO unit. The olefin stream may be at a temperature of about 60° C. (140° F.) to about 150° C. (302° F.), preferably about 80° C. (176° F.) to about 100° C. (212° F.) and a pressure of about 3.5 MPag (500 psig) to about 8.4 MPag (1200 psig).

The olefin stream may be initially contacted with a first-stage oligomerization catalyst to oligomerize the ethylene to oligomers and then contacted with a second-stage oligomerization catalyst to further oligomerize ethylene oligomers and unreacted ethylene. The oligomerization of ethylene generates a large exotherm. For example, dimerization of ethylene can generate 612 kcal/kg (1100 BTU/lb) of heat. Consequently, this large exotherm must be managed. Whereas dimerization of butene can generate 222 kcal/kg (400 BTU/lb) of heat.

Accordingly, the olefin stream in line 210 may feed a header 233 which may split the olefin stream into multiple olefin streams. In FIG. 3, the olefin stream is split into four separate streams: a first olefin stream in charge line 232a, a second olefin stream in charge line 232b, a third olefin stream in charge line 232c and a fourth or last olefin stream in charge line 232d. The header 233 may transport the olefin stream from line 210 to lines 232b, 232c and 232d. In an embodiment, no flow may be permitted through the control valve on a charge line 232d or even on a charge line 232c, so the charge olefin stream is only fed to the upstream first stage oligomerization catalyst beds 242a-242c or just 242a-242b, respectively. More or less separate multiple olefin streams may be used. Up to six olefin streams are readily contemplated. The charge olefin stream in line 210 may be split into equal aliquot multiple olefin streams. Alternatively, the charge olefin stream in line 210 may be split into unequal streams. For example, the charge olefin stream may be split into streams of ascending flow rates in which a subsequent olefin stream has a larger flow rate than a preceding stream. In an embodiment, the charge olefin stream is split into three streams of a predominance of the flow rate of the charge olefin stream from line 210 such as about 40 to about 60% in line 232a and less than a predominance of the flow rate of the charge olefin stream from line 210 in lines 232b and 232c, such as about 15 to about 40%, and no flow in line 232d. Preferably, more of the flow rate of the charge olefin stream is fed to the upstream bed 242a of the upstream first stage oligomerization catalyst beds 242a to 242c. In an embodiment, the charge olefin stream from line 210 may be split into just lines 232a and 232b, perhaps equally. Furthermore, all of the charge olefin stream in line 210 may be directed through charge line 232a.

To manage the exotherm, the olefin stream may be diluted with a diluent stream to provide a diluted olefin stream to absorb the exotherm. The diluent stream may comprise a paraffin stream in a diluent line 234. The diluent stream in the diluent line 234 may be added to the charge olefin stream in line 210 before the charge olefin stream is split into multiple olefin streams. Preferably, the diluent stream is added to the first olefin stream in line 232a after the split into multiple olefin streams to provide a first diluted olefin stream in line 236a, so the diluent stream passes through all of oligomerization reactions. Alternatively, the diluent stream may also be split into multiple streams with each diluent stream added to a corresponding olefin stream. The diluent stream may have a volumetric flow rate of about 2 to about 8 times and preferably about 3 to about 6 times the mass flow rate of the charge olefin stream. The first diluted olefin stream may comprise no more than 17 wt % olefins, suitably no more than 10 wt % olefins and preferably no more than 6 wt % olefins. The first diluted olefin stream may comprise no more than 14 wt % ethylene, suitably no more than 10 wt % ethylene and preferably no more than 6 wt % ethylene. The first diluted olefin stream in line 236a may be cooled in a first charge cooler 238a to provide a first cooled diluted charge olefin stream in line 240a and charged to a first bed 242a of first stage oligomerization catalyst in a first stage oligomerization reactor 242. The cooled diluted first charge olefin stream in line 240a may be charged at a temperature of about 180° C. (356° F.) to about 260° C. (500° F.) and a pressure of about 3.5 MPag (500 psig) to about 8.4 MPag (1200 psig). The charge cooler 238a may comprise a steam generator.

The first stage oligomerization reactor 242 may comprise a series of first stage oligomerization catalyst beds 242a, 242b, 242c and 242d for charging with each multiple olefin stream 232a, 232b, 232c, and 232d, respectively. As earlier stated, in an embodiment, no charge olefin stream may be in line 232d, so the charge olefin stream from line 210 is only fed to the upstream first stage oligomerization catalyst beds 232a-232c. Alternatively, as also earlier stated, in an embodiment, no charge olefin stream may be in lines 232c or 232d, so the charge olefin stream from line 210 is only fed to the upstream first stage oligomerization catalyst beds 232a and 232b. The first stage oligomerization reactor preferably contains four fixed oligomerization catalyst beds 242a, 242b, 242c and 242d. It is also contemplated that each oligomerization catalyst bed 242a, 242b, 242c and 242d may be in a dedicated first stage oligomerization reactor or multiple first stage oligomerization catalyst beds may be in two or more separate first stage oligomerization reactors. Up to six, first stage oligomerization catalyst beds are readily contemplated. A parallel first stage oligomerization reactor may be used when the first stage oligomerization reactor 242 has deactivated during which the first stage oligomerization reactor 242 is regenerated in situ by combustion of coke from the catalyst as will be described hereinafter.

A recycle olefin stream in line 246 may be charged to the oligomerization catalyst beds 242a-242d. In an embodiment, the recycle olefin stream in line 246 is only fed to the downstream oligomerization catalyst beds 242c and 242d. The recycle olefin stream in line 246 may be split into multiple recycle olefin streams in lines 246c and 246d and only fed to oligomerization catalyst beds 242c and 242d, respectively. In an embodiment, line 246c may have a predominance of the flow rate and 246d may have the remainder of flow rate of the recycle olefin stream in line 246. Preferably, more of the flow rate of the recycle olefin stream is fed to the downstream catalyst bed 242d of the downstream catalyst beds 242c and 242d to achieve similar light olefin concentration for managing the exotherm.

The first cooled, diluted olefin stream may be charged to an upstream first, first stage oligomerization catalyst bed 242a in line 240a preferably in a down flow operation. However, upflow operation may be suitable. As oligomerization of ethylene occurs in the upstream first, first stage oligomerization catalyst bed 242a, an exotherm is generated due to the exothermic nature of the ethylene and/or propylene oligomerization reactions. Oligomerization of the first olefin stream produces a first oligomerized olefin stream in an upstream first oligomerized effluent line 244a at an elevated outlet temperature despite the cooling and dilution. The elevated outlet temperature is limited to between 25° C. (45° F.) and about 61° C. (110° F.) above the inlet temperature to the upstream first, first stage catalyst bed 242a.

The second olefin stream in line 232b may receive a split of the charge olefin stream from line 210 from the header 233 governed by the valve on line 232b. The second olefin stream in line 232b may be diluted with the first oligomerized olefin stream in the upstream first oligomerized effluent line 244a removed from the upstream first, first stage oligomerization reactor 242a to provide a second diluted olefin stream in line 236b. The upstream first oligomerized olefin stream in line 244a includes the diluent stream from diluent line 234 added to the first olefin stream in line 232a. The second diluted olefin stream may comprise no more than 22 wt % olefins, suitably no more than 15 wt % olefins and preferably no more than 10 wt % olefins. The second diluted olefin stream may comprise no more than 15 wt % ethylene, suitably no more than 10 wt % ethylene and preferably no more than 6 wt % ethylene. The second diluted olefin stream in line 236b may be cooled in a second charge cooler 238b which may be located externally to the downstream first, first stage oligomerization reactor 242 to provide a second cooled diluted olefin stream in line 240b and charged to a downstream first, first stage bed 242b of oligomerization catalyst in a first stage oligomerization reactor 242. The charge cooler 238b may comprise a steam generator. The second cooled diluted olefin stream in line 240b may be charged at a temperature of about 180° C. (356° F.) to about 260° C. (500° F.) and a pressure of about 3.5 MPag (500 psig) to about 8.4 MPag (1200 psig). The second diluted olefin stream will include diluent and olefins from the first oligomerized olefin stream. The olefins from the first oligomerized olefin stream will oligomerize in the downstream first, first stage bed catalyst bed 242b. Oligomerization of ethylene, propylene and oligomers in the second olefin stream in the downstream first, first stage bed 242b of oligomerization catalyst produces a downstream first, first stage oligomerized olefin stream in a downstream first oligomerized effluent line 244b at an elevated outlet temperature. The elevated outlet temperature may be limited to between 25° C. (45° F.) and about 61° C. (110° F.) above the inlet temperature to the catalyst bed 242b.

The third olefin stream in line 232c may receive a split of the charge olefin stream from line 210 through the header 233 governed by the valve on line 232c. The third olefin stream in line 232c may be diluted with the downstream first, first stage oligomerized olefin stream in line 244b removed from the second, first stage oligomerization reactor 242b and mixed with a first recycle olefin stream in line 246c to provide an upstream, second first stage diluted olefin stream in line 236c. The downstream first stage oligomerized olefin stream in line 244b includes the diluent stream from diluent line 234 added to the first olefin stream in line 232a. The third diluted olefin stream may comprise no more than 23 wt % olefins, suitably no more than 15 wt % olefins and preferably no more than 10 wt % olefins. The third diluted olefin stream may comprise no more than 13 wt % ethylene, suitably no more than 10 wt % ethylene and preferably no more than 6 wt % ethylene. The third diluted olefin stream in line 236c may be cooled in a third charge cooler 238c which may be located externally to the first stage oligomerization reactor 242 to provide a third cooled diluted olefin stream in line 240c and charged to a third bed 242c of oligomerization catalyst in the first stage oligomerization reactor 242. The charge cooler 238c may comprise a steam generator. The third cooled diluted olefin stream in line 240c may be charged at a temperature of about 180° C. (356° F.) to about 260° C. (500° F.) and a pressure of about 3.5 MPag (500 psig) to about 8.4 MPag (1200 psig). The third diluted olefin stream will include diluent and olefins from the downstream second, first stage oligomerized olefin stream and from the first recycle olefin stream. The olefins from the downstream first, first stage oligomerized olefin stream and the first recycle olefin stream will oligomerize in the upstream second, first stage catalyst bed 242c. Oligomerization of ethylene in the third diluted olefin stream in the upstream second, first stage bed 242c of oligomerization catalyst produces an upstream second, first stage oligomerized olefin stream in an upstream second, first stage oligomerized effluent line 244c at an elevated outlet temperature. In an embodiment, the upstream second, first stage oligomerized olefin stream is a penultimate oligomerized olefin stream and the upstream second, first stage oligomerized effluent line 244c is a penultimate oligomerized effluent line 244c. The elevated outlet temperature is limited to between 25° C. (45° F.) and about 61° C. (110° F.) above the inlet temperature to the catalyst bed 242c.

The fourth olefin stream in line 232d may receive a split of the charge olefin stream from line 210 through the header 233 governed by the valve on line 232d. The fourth olefin stream in line 232d may be diluted with the upstream second, first stage or penultimate oligomerized olefin stream in line 244c removed from the upstream second, first stage oligomerization reactor 242 and the second recycle olefin stream in line 246d to provide a fourth diluted olefin stream in line 236d. The upstream second, first stage or penultimate oligomerized olefin stream in line 244c includes the diluent stream from diluent line 234 added to the first olefin stream in line 232a. The fourth diluted olefin stream may comprise no more than 24 wt % olefins, suitably no more than 18 wt % olefins and preferably no more than 10 wt % olefins. The fourth diluted olefin stream may comprise no more than 11 wt % ethylene, suitably no more than 8 wt % ethylene and preferably no more than 6 wt % ethylene. The fourth diluted olefin stream in line 236d may be cooled in a fourth charge cooler 238d which may be located externally to the first stage oligomerization reactor 242 to provide a fourth cooled diluted olefin stream in line 240d and charged to a downstream second, first stage bed 242d of oligomerization catalyst in the first stage oligomerization reactor 242. The charge cooler 238d may comprise a steam generator. The fourth cooled diluted olefin stream in line 240d may be charged at a temperature of about 180° C. (356° F.) to about 260° C. (500° F.) and a pressure of about 3.5 MPag (500 psig) to about 8.4 MPag (1200 psig). The fourth or last diluted olefin stream will include diluent and olefins from the upstream, second, first stage or penultimate oligomerized olefin stream and the second recycle olefin stream. The olefins from the upstream, second, first stage or penultimate oligomerized olefin stream and the second recycle olefin stream will oligomerize in the downstream, second, first stage catalyst bed 242d. Oligomerization of ethylene in the fourth olefin stream in the downstream second, first stage bed 242d of oligomerization catalyst produces a downstream, second first stage oligomerized olefin stream in a fourth oligomerized effluent line 244d at an elevated outlet temperature. The elevated outlet temperature is limited to between 25° C. (45° F.) and about 61° C. (110° F.) above the inlet temperature to the downstream second, first stage catalyst bed 242d.

The recycle olefin stream 246c and 246d provide olefins that can oligomerize over the catalyst beds 242c and 242d, respectively, but can also help manage the exotherm generated during oligomerization of ethylene.

In an embodiment, the downstream second, first stage oligomerized olefin stream is a last olefin stream, and the downstream second, first stage oligomerized effluent line 244d is a last oligomerized effluent line 244d.

The oligomerization reaction takes place predominantly in the liquid phase or in a mixed liquid and gas phase at a LHSV 0.5 to 10 hr¹ on an olefin basis. We have found that a predominant fraction of ethylene in the olefin stream converts to higher olefins. Typically, at least 20 to about 40 mol % of ethylene will oligomerize across an oligomerization catalyst bed. The ethylene will initially oligomerize over the catalyst to butenes.

The first stage oligomerization catalyst may include a zeolitic catalyst. The first stage oligomerization catalyst may be considered a solid acid catalyst. The zeolite may comprise between about 5 and about 95 wt % of the catalyst, for example between about 5 and about 85 wt %. Suitable zeolites include zeolites having a structure from one of the following classes: MFI, MEL, ITH, IMF, TUN, FER, BEA, FAU, BPH, MEI, MSE, MWW, UZM-8, MOR, OFF, MTW, TON, MTT, AFO, ATO, and AEL. 3-letter codes indicating a zeotype are as defined by the Structure Commission of the International Zeolite Association and are maintained at http://www.iza-structure.org/databases. UZM-8 is as described in U.S. Pat. No. 6,756,030. In a preferred aspect, the first stage oligomerization catalyst may comprise a zeolite with a framework having a ten-ring pore structure. Examples of suitable zeolites having a ten-ring pore structure include TON, MTT, MFI, MEL, AFO, AEL, EUO and FER. In a further preferred aspect, the first stage oligomerization catalyst comprising a zeolite having a ten-ring pore structure may comprise a uni-dimensional pore structure. A uni-dimensional pore structure indicates zeolites containing non-intersecting pores that are substantially parallel to one of the axes of the crystal. The pores preferably extend through the zeolite crystal. Suitable examples of zeolites having a ten-ring uni-dimensional pore structure may include MTT. In a further aspect, the first stage oligomerization catalyst comprises an MTT zeolite.

The first stage oligomerization catalyst may be formed by combining the zeolite with a binder, and then forming the catalyst into pellets. The pellets may optionally be treated with a phosphorus reagent to create a zeolite having a phosphorous component between 0.5 and 15 wt % of the treated catalyst. The binder is used to confer hardness and strength on the catalyst. Binders include alumina, aluminum phosphate, silica, silica-alumina, zirconia, titania and combinations of these metal oxides, and other refractory oxides, and clays such as montmorillonite, kaolin, palygorskite, smectite and attapulgite. A preferred binder is an aluminum-based binder, such as alumina, aluminum phosphate, silica-alumina and clays.

One of the components of the catalyst binder utilized in the present disclosure is alumina. The alumina source may be any of the various hydrous aluminum oxides or alumina gels such as alpha-alumina monohydrate of the boehmite or pseudo-boehmite structure, alpha-alumina trihydrate of the gibbsite structure, beta-alumina trihydrate of the bayerite structure, and the like. A suitable alumina is available from UOP LLC under the trademark VERSAL. This material is an extremely high purity alpha-alumina monohydrate (pseudo-boehmite) which after calcination at a high temperature has been shown to yield a high purity gamma-alumina.

A suitable first stage oligomerization catalyst is prepared by mixing proportionate volumes of zeolite and alumina to achieve the desired zeolite-to-alumina ratio. In an embodiment, the MTT content may about 5 to about 85, for example about 20 to about 82 wt % MTT zeolite, and the balance alumina powder will provide a suitably supported catalyst. A silica support is also contemplated.

Monoprotic acid such as nitric acid or formic acid may be added to the mixture in aqueous solution to peptize the alumina in the binder. Additional water may be added to the mixture to provide sufficient wetness to constitute a dough with sufficient consistency to be extruded or spray dried. Extrusion aids such as cellulose ether powders can also be added. A preferred extrusion aid is available from The Dow Chemical Company under the trademark Methocel.

The paste or dough may be prepared in the form of shaped particulates, with the preferred method being to extrude the dough through a die having openings therein of desired size and shape, after which the extruded matter is broken into extrudates of desired length and dried. A further step of calcination may be employed to give added strength to the extrudate. Generally, calcination is conducted in a stream of air at a temperature from about 260° C. (500° F.) to about 815° C. (1500° F.). The MTT catalyst is not selectivated to neutralize acid sites such as with an amine.

The extruded particles may have any suitable cross-sectional shape, i.e., symmetrical or asymmetrical, but most often have a symmetrical cross-sectional shape, preferably a spherical, cylindrical or polylobal shape. The cross-sectional diameter of the particles may be as small as m. However, it is usually about 0.635 mm (0.25 inch) to about 12.7 mm (0.5 inch), preferably about 0.79 mm ( 1/32 inch) to about 6.35 mm (0.25 inch), and most preferably about 0.06 mm ( 1/24 inch) to about 4.23 mm (⅙ inch).

In one exemplary embodiment, an MTT-type zeolite catalyst disposed on a high purity pseudo boehmite alumina substrate in a ratio of about 90/10 to about 20/80 and preferably between about 20/80 and about 50/50 is provided in a catalyst bed or more in the first stage oligomerization reactor 242.

The zeolite catalyst is advantageous as a first stage oligomerization catalyst. The zeolitic catalyst has relatively low sensitivity towards oxygenates contamination. Consequently, less removal of oxygenates is required of olefinic feed in line 232 if produced from an ethanol dehydration process.

The last oligomerized olefin stream in the last oligomerized effluent line 244d has an increased concentration of ethylene dimers and oligomers compared to the charge olefin stream in line 232. The oligomerized olefin stream is cooled to generate steam in a steam generator 247, then cooled by heat exchange with an oligomerized stream in line 257 in a heat exchanger 249 and then cooled in an air cooler 251 before it is charged to a second stage oligomerization reactor 252 in an oligomerization charge line 248. The second stage oligomerization reactor 252 may comprise a series of second stage oligomerization catalyst beds 252a and 252b in series. It is also contemplated that each second stage oligomerization catalyst bed 252a and 252b may be in a dedicated second stage oligomerization reactor or multiple second stage oligomerization catalyst beds may be in two or more separate second stage oligomerization reactors. More than two second stage oligomerization catalyst beds are readily contemplated. A parallel second stage oligomerization reactor may be used when the second stage oligomerization reactor 252 has deactivated during which the second stage oligomerization reactor 252 is regenerated in situ by combustion of coke from the catalyst.

To achieve the most desirable olefin product, the second stage oligomerization reactor 252 is operated at a temperature from about 38° C. (100° F.) to about 180° C. (356° F.). The second stage oligomerization reactor 252 is run at a pressure of about 4.9 MPa (700 psig) to about 7.6 MPa (1100 psig), and more preferably from about 3.5 MPa (500 psig) to about 6.9 MPa (1000 psig). A first oligomerate stream from the first second stage oligomerization catalyst bed 252a may be withdrawn from the first, second stage oligomerization reactor 252 in line 253, cooled in a cooler 254 back to a temperature of about 38° C. (100° F.) to about 180° C. (356° F.) and charged to the second, second stage oligomerization catalyst bed 252b in line 255.

The second stage oligomerization reactor 252 may be in downstream communication with the first stage oligomerization reactor 242. The second stage oligomerization reactor 252 preferably operates in a down flow operation. However, upflow operation may be suitable. The charge oligomerized olefin stream is contacted with the second stage oligomerization catalyst causing the C2-C8 olefins to dimerize and trimerize to provide distillate range olefins. With regard to the second stage oligomerization reactor 252, process conditions are selected to produce a higher percentage of jet range olefins which, when hydrogenated in a subsequent step as will be described below, result in a desirable jet-range hydrocarbon product. A predominance of the unconverted ethylene in the charge oligomerized olefin stream is oligomerized in the second stage oligomerization reactor 252. In an embodiment, at least 90 mol % of ethylene in the charge oligomerized olefin stream is oligomerized in the second stage oligomerization reactor 252. The metal, second stage oligomerization catalyst is efficient at dimerizing un-dimerized ethylene. An oligomerate olefin stream with an increased average carbon number greater than the oligomerized olefin stream charged in the oligomerization charge line 248 exits the oligomerization reactor 252 in line 257.

The second stage oligomerization catalyst is preferably an amorphous silica-alumina base with a metal from either Group VIII and/or Group VIB in the periodic table using Chemical Abstracts Service notations. In an aspect, the catalyst has a Group VIII metal promoted with a Group VIB metal. Typically, the silica and alumina will only be in the base, so the silica-to-alumina ratio will be the same for the catalyst as for the base. The added metals can either be impregnated onto or ion exchanged with the silica-alumina base. Co-mulling is also contemplated. Catalysts for the present invention may have a Low Temperature Acidity Ratio of at least about 0.15, suitably of about 0.2, and preferably greater than about 0.25, as determined by Ammonia Temperature Programmed Desorption (Ammonia TPD) as described hereinafter. Additionally, a suitable second stage oligomerization catalyst will have a surface area of between about 50 and about 400 m²/g as determined by nitrogen BET.

The preferred second stage oligomerization catalyst comprises an amorphous silica-alumina support. One of the components of the catalyst support utilized in the present invention is alumina. The alumina may be any of the various hydrous aluminum oxides or alumina gels such as alpha-alumina monohydrate of the boehmite or pseudo-boehmite structure, alpha-alumina trihydrate of the gibbsite structure, beta-alumina trihydrate of the bayerite structure, and the like. A particularly preferred alumina is available from Sasol North America Alumina Product Group under the trademark Catapal. This material is an extremely high purity alpha-alumina monohydrate (pseudo-boehmite) which after calcination at a high temperature has been shown to yield a high purity gamma-alumina. Another component of the catalyst support is an amorphous silica-alumina. A suitable silica-alumina with a silica-to-alumina ratio of 2.6 is available from CCIC, a subsidiary of JGC, Japan.

Another component utilized in the preparation of the second stage oligomerization catalyst utilized in the present invention is a surfactant. The surfactant is preferably admixed with the hereinabove described alumina and the silica-alumina powders. The resulting admixture of surfactant, alumina and silica-alumina is then formed, dried and calcined as hereinafter described. The calcination effectively removes by combustion the organic components of the surfactant but only after the surfactant has dutifully performed its function in accordance with the present invention. Any suitable surfactant may be utilized. A preferred surfactant is a surfactant selected from a series of commercial surfactants sold under the trademark “Antarox” by Solvay S.A. The “Antarox” surfactants are generally characterized as modified linear aliphatic polyethers and are low-foaming biodegradable detergents and wetting agents.

A suitable silica-alumina mixture is prepared by mixing proportionate volumes silica-alumina and alumina to achieve the desired silica-to-alumina ratio. In an embodiment, about 75 to about 95 wt-% amorphous silica-alumina with a silica-to-alumina ratio of 2.6 and about 10 to about 20 wt-% alumina powder will provide a suitable support. In an embodiment, other ratios of amorphous silica-alumina to alumina may be suitable.

Any convenient method may be used to incorporate a surfactant with the silica-alumina and alumina mixture. The surfactant is preferably admixed during the admixture and formation of the alumina and silica-alumina. A preferred method is to admix an aqueous solution of the surfactant with the blend of alumina and silica-alumina before the final formation of the support. It is preferred that the surfactant be present in the paste or dough in an amount from about 0.01 to about 10 wt-% based on the weight of the alumina and silica-alumina.

Monoprotic acid such as nitric acid or formic acid may be added to the mixture in aqueous solution to peptize the alumina in the binder. Additional water may be added to the mixture to provide sufficient wetness to constitute a dough with sufficient consistency to be extruded or spray dried.

The paste or dough may be prepared in the form of shaped particulates, with the preferred method being to extrude the dough mixture of alumina, silica-alumina, surfactant and water through a die having openings therein of desired size and shape, after which the extruded matter is broken into extrudates of desired length and dried. A further step of calcination may be employed to give added strength to the extrudate. Generally, calcination is conducted in a stream of dry air at a temperature from about 260° C. (500° F.) to about 815° C. (1500° F.).

The extruded particles may have any suitable cross-sectional shape, i.e., symmetrical or asymmetrical, but most often have a symmetrical cross-sectional shape, preferably a spherical, cylindrical or polylobal shape. The cross-sectional diameter of the particles may be as small as m. However, it is usually about 0.635 mm (0.25 inch) to about 12.7 mm (0.5 inch), preferably about 0.79 mm ( 1/32 inch) to about 6.35 mm (0.25 inch), and most preferably about 0.06 mm ( 1/24 inch) to about 4.23 mm (⅙ inch).

Typical characteristics of the amorphous silica-alumina supports utilized herein are a total pore volume, average pore diameter and surface area large enough to provide substantial space and area to deposit the active metal components. The total pore volume of the support, as measured by conventional mercury porosimeter methods, is usually about 0.2 to about 2.0 cc/gram, preferably about 0.25 to about 1.0 cc/gram and most preferably about 0.3 to about 0.9 cc/gram. Ordinarily, the amount of pore volume of the support in pores of diameter greater than 100 angstroms is less than about 0.1 cc/gram, preferably less than 0.08 cc/gram, and most preferably less than about 0.05 cc/gram. Surface area, as measured by the B.E.T. method, is typically above 50 m²/gram, e.g., above about 200 m²/gram, preferably at least 250 m²/gram, and most preferably about 300 m²/gram to about 400 m²/gram.

To prepare the catalyst, the support material is compounded, as by a single impregnation or multiple impregnations of a calcined amorphous refractory oxide support particles, with one or more precursors of at least one metal component from Group VIII or VIB of the periodic table. The Group VIII metal, preferably nickel, should be present in a concentration of about 0.5 to about 15 wt-% and the Group VIB metal, preferably tungsten, should be present in a concentration of about 0 to about 12 wt-%. The impregnation may be accomplished by any method known in the art, as for example, by spray impregnation wherein a solution containing the metal precursors in dissolved form is sprayed onto the support particles. Another method is the multi-dip procedure wherein the support material is repeatedly contacted with the impregnating solution with or without intermittent drying. Yet other methods involve soaking the support in a large volume of the impregnation solution or circulating the support therein, and yet one more method is the pore volume or pore saturation technique wherein support particles are introduced into an impregnation solution of volume just sufficient to fill the pores of the support. On occasion, the pore saturation technique may be modified, so as to utilize an impregnation solution having a volume between 10 percent less and 10 percent more than that which will just fill the pores.

If the active metal precursors are incorporated by impregnation, a subsequent or second calcination at elevated temperatures, as for example, between 399° C. (750° F.) and 760° C. (1400° F.), converts the metals to their respective oxide forms. In some cases, calcinations may follow each impregnation of individual active metals. A subsequent calcination yields a catalyst containing the active metals in their respective oxide forms.

A preferred second stage oligomerization catalyst of the present invention has an amorphous silica-alumina base impregnated with 0.5-15 wt-% nickel in the form of 3.175 mm (0.125 inch) extrudates and a density of about 0.45 to about 0.65 g/ml. It is also contemplated that metals can be incorporated onto the support by other methods such as ion-exchange and co-mulling.

Second stage oligomerization reactions are also exothermic in nature. The last oligomerized olefin stream in line 244d includes the diluent stream from diluent line 234 added to the first olefin stream in line 232a and carried through the first stage oligomerization catalyst beds 242a-242d. The diluent stream is then transported into the second stage oligomerization reactor 252 in line 248 to absorb the exotherm in the second stage oligomerization reactor. It is contemplated to introduce diluent to the second stage oligomerization catalyst beds 252a and 252b with or without it first passing through the first stage oligomerization catalyst beds.

When the oligomerization reaction is performed according to the above-noted process conditions, a C4 olefin conversion of greater than or equal to about 95% is achieved, or greater than or equal to 97%. The resulting oligomerate olefin stream in line 257 includes a plurality of olefin products that are distillate range hydrocarbons.

An oligomerate olefin stream in line 257 with an increased C9+ olefin concentration compared to the oligomerized olefin stream in line 248 may be heat exchanged with the last oligomerized olefin stream in line 244d in the heat exchanger 249 and an olefin splitter bottoms stream in line 261 in a heat exchanger 263, let down in pressure, perhaps combined with drained oligomerate in line 259 from a first stage oligomerization reactor 242 or a second stage oligomerization reactor 252 and fed to olefin splitter column 256. The oligomerized olefin stream in line 257 is at a temperature from about 140° C. (284° F.) to about 200° C. (392° F.) and a pressure of about 3.9 MPa (gauge) (550 psig) to about 6.3 MPa (gauge) (900 psig).

In the olefin splitter column 256 oligomers that boil lower than the jet range hydrocarbons, typically C8− hydrocarbons with atmospheric boiling points less than about 150° C., are separated in an olefin splitter overhead stream in an overhead line 258 from a bottoms stream in a bottoms line 260 comprising distillate-range C9+ hydrocarbons, typically C9-C22 olefins. The olefin splitter column 256 may be operated at a bottom temperature of about 200° C. (400° F.) to about 315° C. (600° F.) and an overhead pressure of about 35 kPa (gauge) (5 psig) to about 350 kPa (gauge) (50 psig). It is envisioned that the olefin splitter column 256 may be two columns.

The olefin splitter overhead stream may be cooled to about 66° C. (150° F.) to about 93° C. (200° F.) and a resulting condensate portion refluxed from an olefin splitter receiver 262 back to the olefin splitter column 256. A net vapor stream in a receiver overhead line 264 from the olefin splitter receiver 262 may be compressed up to oligomerization pressure in an off-gas compressor 266 to provide a light oligomer stream in line 268 either in vapor phase or in liquid phase after cooling. Alternatively, the olefin splitter overhead stream in the receiver overhead line 264 may be fully condensed by cooling perhaps in an external refrigeration loop to provide a liquid light oligomer stream in line 268. Line 268 may also be taken from the receiver 262. The light oligomer stream in line 268 may be split between a light olefin drag stream in line 270 and the oligomer recycle stream in line 246 that may be recycled to the second stage oligomerization reactor 252 or to the first stage oligomerization reactor 242. The light olefin drag stream in line 270 may comprise about 1 to about 15 wt % of the light oligomer stream in line 268. The light oligomer stream in line 268 may comprise about 30 to about 80 wt % light olefins.

In an embodiment, the oligomer recycle stream in line 246 may be mixed with the last oligomerized olefin stream in the last dimerized effluent line 244d to provide the charge oligomerization stream in line 248 for charge to the second stage oligomerization reactor 252. The oligomer recycle stream in line 246 may be mixed with the first diluted olefin stream in line 236a or more suitably divided up between the first through fourth diluted olefin streams in lines 236a-236d to oligomerize unreacted C4-C7 olefins. In a preferred embodiment, the oligomer recycle stream in line 246 may be split into a first oligomer recycle stream in line 246c and a second oligomer recycle stream in line 246d. The first oligomer recycle stream in line 246c may be mixed with the upstream second, first stage oligomerized olefin stream in the downstream first, first stage oligomerized effluent line 244b and perhaps the third olefin stream in the third olefin line 232c to provide the third diluted olefin stream in line 236c for charge to the upstream second, first-stage oligomerization catalyst bed 242c. The second oligomer recycle stream in line 246d may be mixed with the upstream second, first stage oligomerized olefin stream in the upstream second, first stage oligomerized effluent line 244c and perhaps the third olefin stream in the third olefin line 232d to provide the fourth diluted olefin stream in line 236d for charge to the downstream second, first-stage oligomerization catalyst bed 242d.

The heavy olefin stream in the splitter bottoms line 260 may be split between a reboil stream in line 271 that is reboiled and fed back to the olefin splitter column 256 and a heavy olefin stream in a net splitter bottoms line 261. The heavy olefin stream in the net bottoms line 261 is cooled by heat exchange with the oligomerized olefin stream in line 257 and then transported to the hydrogenation section 300 in FIG. 4 in line 250. The reboil stream in line 271 may be heated by heat exchange with a reboil stream in line 333 from the jet fractionation bottoms line 326 which is returned to the jet fractionation column in line 335 in FIG. 4.

Turning to the hydrogenation section 300 in FIG. 4, the heavy olefin stream in the net olefin splitter bottoms line 250 from FIG. 3 comprising distillate-range C9+ oligomerized olefins may be hydrogenated to saturate the olefinic bonds in a hydrogenation reactor 302 to provide fuels. This step is performed to ensure the product motor fuel meets or exceeds the thermal oxidation requirements specified in ASTM D7566-10a for hydroprocessed synthesized paraffinic kerosene (SPK). Additionally, saturating the oligomerized heavy olefins will provide the paraffin stream that may be used as the diluent stream in line 234. The heavy olefin stream in line 250 may be cooled to produce steam and be combined with the light olefin drag stream comprising C₂ to C₈ olefins in line 270 also from FIG. 3 to produce a combined olefin stream in line 304. The combined olefin stream in line 304 may also be combined with a hydrogen stream in line 306 to provide a combined hydrogenation charge stream in line 308 which is cooled and charged to the hydrogenation reactor 302 at 125° C. (257° F.) to about 315° C. (600° F.) and 3.5 MPag (500 psig) to about 6.9 MPag (1000 psig). An excess of hydrogen may be employed to ensure complete saturation such as about 1.5 to about 2.5 of stochiometric hydrogen.

Hydrogenation is typically performed using a conventional hydrogenation or hydrotreating catalyst, and can include metallic catalysts containing, e.g., palladium, rhodium, nickel, ruthenium, platinum, rhenium, cobalt, molybdenum, or combinations thereof, and the supported versions thereof. Catalyst supports can be any solid, inert substance including, but not limited to, oxides such as silica, alumina, titania, calcium carbonate, barium sulfate, and carbons. The catalyst support can be in the form of powder, granules, pellets, or the like.

In an exemplary embodiment, hydrogenation is performed in the hydrogenation reactor 302 that includes a platinum-on-alumina catalyst, for example, about 0.5 wt % to about 0.9 wt % platinum-on-alumina catalyst. Nickel-alumina catalyst may also be suitable. The hydrogenation reactor 302 converts the olefins into a paraffin product having the same carbon number distribution as the olefins, thereby forming distillate-range paraffins suitable for use as jet and diesel fuel.

The saturated heavy stream discharged from the hydrogenation reactor 302 in line 310 may be cooled by heat exchange with a saturated heavy liquid stream in a separator bottoms line 316 and fed to a hydrogenation separator 312. In the hydrogenation separator 312, the saturated heavy stream is separated into a hydrogenated separator vapor stream in an overhead line 314 and the saturated heavy liquid stream in the hydrogenation separator bottoms line 316. A purge in line 315 may be taken from the hydrogenated separator vapor stream in line 314 and the remainder may be compressed and combined with make-up hydrogen in line 318 to provide the hydrogen stream in line 306. The saturated heavy liquid stream in the bottoms line 316 may be heated by heat exchange with the saturated heavy stream in line 310 and the diluent stream in line 234 and fed to a jet fractionation column 320. In an embodiment, a hot separator and cold separator system may be substituted for a single hydrogenation separator 312.

The saturated heavy liquid stream in the bottoms line 316 may be fed to the jet fractionation column 320 without undergoing prior stripping in a stripper column. Alternatively, a stripper column may be utilized upstream of the jet fractionation column 320. In the jet fractionation column 320, the saturated heavy liquid stream may be separated into a net off-gas stream in a net overhead line 330, a green jet stream in a net liquid overhead line 324 and a green diesel stream in a net bottoms line 332. The jet fractionation column 320 may be operated at a bottom temperature of about 316° C. (600° F.) to about 482° C. (900° F.) and an overhead pressure of about 35 kPa (5 psig) to about 350 kPa (50 psig).

A jet fractionation overhead stream in the overhead line 322 may be cooled to produce a condensate stream in line 329 from a bottom of the receiver 328. A portion of the condensate in line 329 is refluxed back to the jet fractionation column 320 while a jet fuel stream in line 334 is transported to a jet stripper column 340. The net off gas stream comprising C8-hydrocarbons is taken in a receiver overhead line 330 from the jet fractionation receiver 328. Most of the hydrocarbons in the net off gas stream in the receiver overhead line 330 are lighter hydrocarbons and can be used to fuel the reboiler for the jet fractionation column 320, the olefin splitter column 36 and/or another fired heater in the complex.

The jet stripper column 340 strips light ends from the jet fuel stream in line 334 and sends them in the overhead line 342 back to the condenser 323 with the jet fractionation overhead stream in line 322. A stripped jet fuel product is taken in the jet stripper bottoms line 344 while a portion is reboiled and fed back to the jet stripper column 340. A jet fuel product stream in line 324 is taken from the stripped jet fuel product in line 344, cooled in the cooler 345 and recovered as jet fuel product. The green jet stream taken in the line 324 comprises kerosene range C9-C17 hydrocarbons and may be cooled and taken as product meeting applicable SPK standards. In an alternative embodiment, the green jet stream may be taken from a side line from the side of the jet fractionation column 320.

The green diesel bottoms stream in the bottoms line 326 may be split between a reboil stream in line 331 that is reboiled and fed back to the jet fractionation column 320, a green diesel product stream in line 332 and a diluent stream in line 234. The diluent stream in line 234 may be cooled by heat exchange with the separator bottoms line 316 and by steam generation and recycled back to be mixed with the olefin stream in line 232 in the oligomerization section 10 in FIG. 3, preferably the first olefin stream in line 232a, to provide the first diluted olefin stream in line 236a to absorb the exotherm in the first stage oligomerization reactor 242 and the second stage oligomerization reactor 252. The green diesel in the diluent line 234 is paraffinic, so it will be inert to the oligomerization and hydrogenation reactions to which it may be subject. Both the jet fuel stream in the line 324 and the diesel stream in line 332 can be cooled and fed to their respective fuel pools. The diesel stream will meet ASTM D975 standards for diesel. The reboil stream in line 331 may be diverted in line 333 to the olefin splitter column 320 to reboil the reboil stream in line 301 from the olefin splitter bottoms stream in line 260 in FIG. 3. The cooled reboil stream may be returned in line 335 and heated in the heater to boiling and returned to the jet fractionation column 320.

Starting with biomass, the disclosed process can efficiently produce green jet fuel and green diesel fuel that meets applicable fuel requirements while managing exothermic heat generation. Carbon recovery in the process can exceed 95%.

At the end of the 14 to 50 days, preferably at least after 30 days, after the maximum temperature is reached to compensate for declining catalytic activity, the catalytic cycle in the first stage oligomerization reactor 242 and/or the second stage oligomerization reactor 252 must restart. The catalysts may be regenerated to recover their initial activity.

A first stage oligomerization reactor 242 comprising a first, first stage oligomerization catalyst bed 242a and a second, first stage oligomerization catalyst bed 242b are shown in FIG. 5 in a regeneration mode. The regeneration of the zeolitic oligomerization catalyst in the first stage oligomerization reactor 242 is conducted by taking the first stage oligomerization reactor 242 offline by discontinuing olefin charge thereto by closing the valves on line 240a, line 244a and line 244b. Valves on lines 103, 428, 245 and 416 are opened. The regeneration outlet line 245 feeds lines 425 and 416. With valves on lines 245 and 416 opened, the first stage oligomerization reactor 242 is drained of liquid through a regeneration outlet line 245 via a valve thereon. A vapor-liquid mixture rushes to a drum 418 due to high differential pressure. Liquid is pumped to the olefin splitter column 256 in FIG. 3 via line 259 through a valve thereon. The first stage oligomerization reactor 242 reactor is depressurized to about 280 kPa (g) (40 psig) to about 600 kPa (g) (85 psig) by venting gases comprising ethylene and other light olefins in line 103 perhaps to the first stage ethylene suction drum 116 via line 104 in FIG. 2 When depressurization is complete, the valves on lines 103 and 416 are closed.

The first stage oligomerization reactor 242 is then purged with hot, dry inert gas to strip heavy hydrocarbons, C8 to C21, from the first stage oligomerization catalyst in the catalyst beds 242a and 242b. The valves on a supply line 412 and a return line 425 are opened to open a regeneration circuit 408. Typically, an inert gas such as nitrogen or a saturated hydrocarbon stream may be used as a purge gas. We have discovered that the surplus carbon dioxide stream in line 46 generated by fermentation from FIG. 1 is sufficiently inert to provide an inert gas stream for purging. The surplus carbon dioxide stream in line 46 may be passed to the regeneration circuit 408. In the regeneration circuit 408, gas is passed through a compressor 409, a drier 410 and heated by a heat exchanger 414 and passed in the supply line 412 regulated by a valve thereon to the first stage oligomerization reactor 242. The return line 425 may be used to cycle gas through a valve back through the regeneration circuit 408 to boost the gas to desired temperature and/or pressure, thereby bringing the first stage oligomerization reactor 242 to the appropriate regeneration conditions. With continuous flow of carbon dioxide from line 46, the compressor 409 increases the volumetric flow rate of the total purge gas. When the flow rate of carbon dioxide through the circuit 408 is sufficient, the valve on line 416 is opened to the drain tank 418, and the vent is routed to a carbon dioxide sequestration or utilization unit in line 420 through an open valve thereon. Carbon dioxide utilization may include recycle to line 46 for employment in this regeneration process.

The purge gas may be introduced to the first stage oligomerization reactor 242 perhaps in down flow at about 350 to about 450° C. and about 175 kPa (g) (25 psig) to about 280 kPa (g) (40 psig). These conditions are sufficient to strip light olefins from the catalyst into the liquid phase. Inert gas laden with desorbed oligomerate olefins can be directed in line 416 through an open valve thereon from the regeneration outlet 245 of the oligomerization reactor 242 to a drain drum 418. Carbon dioxide can vent in line 420 from an overhead of the drain drum 418 to be treated, sequestered, employed or vented to the stack. Accumulated stripped oligomerate in the drain drum 418 can be drained in line 259 through an open valve thereon and pumped in line 259 to the olefin splitter column 256 in FIG. 3

When the oligomerization catalyst in the reactor 242 is sufficiently stripped of oligomerate hydrocarbons which may be indicated by a steady liquid level in the drain tank 418, the valve on line 259 is closed. However, the valves on lines 412 and 425 remain open to enable inert gas to flow through the regeneration circuit 408 and pressure the first stage oligomerization reactor 242 up to regeneration pressure and enable sufficient flushing by the inert gas through line 420 with the valve thereon open.

Once the first stage oligomerization reactor reaches regeneration pressure of about 101.3 kPa to about 689.5 kPa (g), inert gas flow from line 46 is terminated by closing the valve thereon. An oxygen supply gas such as air is then routed to the first stage oligomerization reactor 242 by opening the valve on line 424 and the exhaust valve on line 420. The valve on the return line 425 may be kept open to enable the compressor to provide a sufficiently high gas space velocity per volume of catalyst. At the inlet, oxygen concentration should not exceed about 0.2 to about 0.7 mol % and at the outlet, the temperature be no more than 400° C. to about 500° C. Carbon burn can endure for about 10 to about 40 hours. Flue gas comprising carbon dioxide from the carbon burn can be routed through line 416 and vented through the exhaust line 420 and perhaps taken to a carbon monoxide treating system before entering carbon dioxide sequestration or utilization. Carbon dioxide utilization can include recycling a carbon dioxide stream in a recycle line 422 to line 46 to provide carbon dioxide as inert gas for the regeneration process. Recycled carbon dioxide in line 422 can supplement or replace the carbon dioxide from the fermentation process in FIG. 1. Carbon monoxide treatment may include thermal oxidation to carbon dioxide. Once the exotherm from the burn no longer rises, the oxygen concentration can be increased to about 5 mol % to about 10 mol % to prove that carbon is sufficiently combusted in the same temperature range to completely regenerate the catalyst restoring it to full oligomerization activity. The gas space velocity based on the total flow rate ranges from about 400 to about 4000 hr⁻¹ and preferably no more than 1000 hr⁻¹ during coke burn. Once coke burn is completed, the valve on the air supply line 424 is closed.

Residual oxygen can then be purged from the first stage oligomerization reactor by an inert gas purge. Consequently, the valve on the inert gas line 46 is opened to bring carbon dioxide back into the regeneration circuit 408. The oxygen from the circuit 408 is purged through the lines 416 and 420 while carbon dioxide is continuously injected from line 46. The carbon dioxide dilutes the volume of oxygen steadily in the circuit. Inert gas carrying residual oxygen is directed in line 416 from the regeneration outlet 245 of the oligomerization reactor 242 through the valve thereon to the drain drum 418. Carbon dioxide can vent in line 420 from an overhead of the drain drum 418 to be treated in the carbon monoxide treating system and then sequestered, employed or vented to the stack. Once the regeneration circuit 408 is purged of oxygen, the valve on line 416 is closed. The inert gas is cycled to the oligomerization reactor 242 to pressure up or maintain regeneration pressure until the reactor is placed back in oligomerization service.

To prepare the first stage oligomerization reactor 242 for restored oligomerization service after regeneration of the oligomerization catalyst, the valve on line 46 is closed to terminate the inert gas flow into the regeneration circuit 408. Additionally, valves on lines 412 and 428 are closed, while valves on lines 416, 240a, and 244a are opened. Diluent from line 234 through an open valve thereon may be fed to the oligomerization reactor 242 to fill it up with heavy hydrocarbon paraffins after regeneration to push out residual gases through lines 245, 416 and 420 and ready the reactor for oligomerization service. When oligomerization service is continued, the valve on line 245 should be closed and the valve on line 244b opened.

To facilitate regeneration without downtime, a swing bed arrangement may be employed with an alternative first stage oligomerization reactor 242. Alternatively, a lead-lag swing bed arrangement may be employed. Activity and selectivity of the regenerated catalyst is comparable to fresh catalyst.

The regeneration of the second stage oligomerization catalyst in the second stage oligomerization reactor 252 comprising metal on a support may be conducted according to the same foregoing procedure as for the first stage oligomerization reactor. In sum, the flow of oligomerized olefin stream is terminated to the second stage oligomerization reactor 252. The second stage oligomerization catalyst bed 252a, for example, is purged with an inert gas which may be provided from the surplus carbon dioxide stream 46 from FIG. 1. The inert gas removes hydrocarbons from the second stage oligomerization catalyst bed 252a. The second stage oligomerization catalyst bed 252a is then regenerated by contact with an oxygen supply gas. The oxygen can be purged from the second stage oligomerization catalyst bed 252a with the inert carbon dioxide and pressured up to oligomerization pressure. Then the second stage oligomerization catalyst bed 252a can be put back online, perhaps filled with diluent and resupplied with a charge oligomerized olefin stream to the catalyst beds 252a and 252b.

We have developed a process for regenerating both catalyst systems in the first stage oligomerization reactor 242 and the second stage oligomerization reactor 252 which allows for continuous operation.

### EXAMPLES

### Example 1

Spent first stage oligomerization MTT zeolite catalyst and second stage oligomerization nickel on amorphous silica alumina catalyst from a first pilot plant cycle test contacted in a stacked bed configuration with ethylene feed and light paraffin diluent were regenerated ex-situ. The regeneration procedure consisted of a high temperature nitrogen purge at 400° C., at 600-800 h⁻¹ gas hourly space velocity, followed by coke burn at 450° C. in a controlled O₂ environment of 2 wt % and 7 wt % O₂ in N₂ and proof coke burn in 18 wt % O₂ in N₂. As shown in Table 1, the 400° C. nitrogen purge removed significant amounts of carbonaceous species. Based on gas chromatography analysis, the purged hydrocarbon species are C8-C20 olefins. The carbon burn step removed completely the coke on the second stage Ni/ASA catalyst. The regenerated first stage zeolite MTT catalyst exhibited sufficient catalyst performance.

TABLE 1
Carbon, hydrogen and nitrogen analysis for spent and hot nitrogen
purged 1^{st} stage and 2^{nd} stage oligomerization catalysts.

| | | | 1st stage | | | |
|---|---|---|---|---|---|---|
| | | | MTT | 2nd stage | Regenerated | |
| | Spent 1st | | zeolite | Ni/ASA | 1st stage | Regenerated |
| | Stage | Spent | catalyst | Catalyst | MTT | 2nd stage |
| | MTT | 2nd stage | after | after | zeolite | Ni/ASA |
| Catalyst | zeolite | Ni/ASA | 400° C. | 400° C. | catalyst | catalyst |
| Description | catalyst | catalyst | N2 Purge | N2 purge | after C burn | after C burn |
| C, wt % | 5.0 | 10.5 | 1.3 | 0.7 | 0.9 | 0.1 |
| H, wt % | 1.05 | 1.87 | 0.49 | 0.38 | 0.75 | 0.66 |
| N, wt % | 0.06 | <0.05 | <0.05 | <0.05 | <0.05 | <0.05 |

### Example 2

Pilot plant tests with both a first stage oligomerization catalyst comprising MTT zeolite and a second stage oligomerization catalysts comprising nickel on amorphous silica alumina confirmed two weeks or longer operation providing satisfactory conversion at acceptable temperatures in a first cycle with complete performance recovery in the second cycle after ex-situ regeneration.

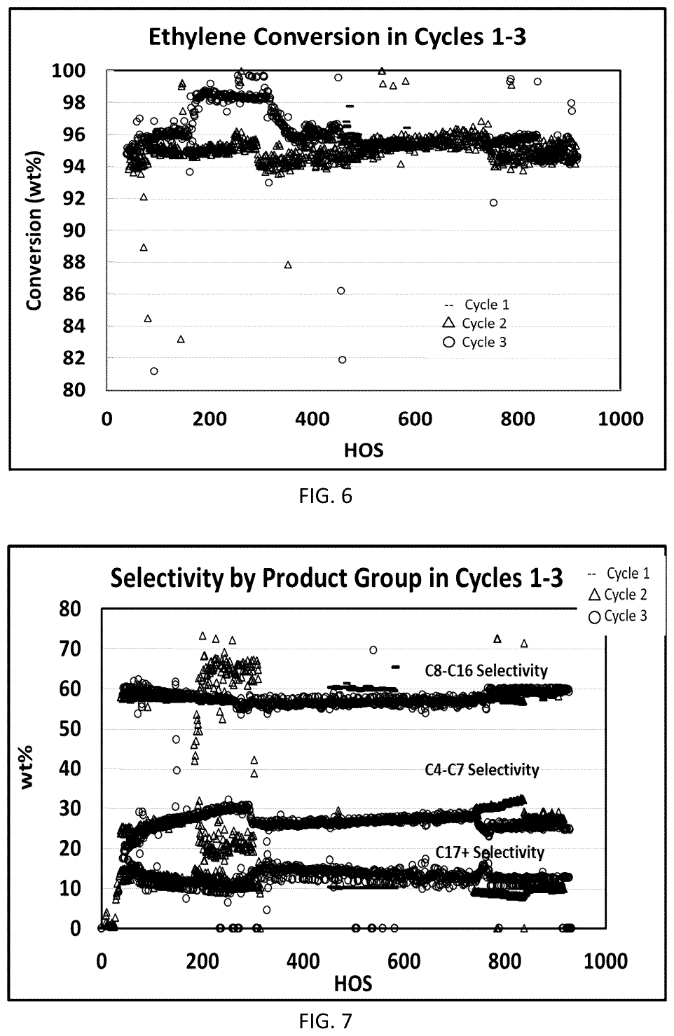

FIG. 6 shows ethylene conversion over time for three cycles. The first cycle is with fresh MTT zeolite catalyst and nickel on amorphous silica alumina catalyst arranged in a stacked bed configuration in the pilot plant reactor indicated by the dashes. The second cycle in triangles and the third cycle in circles represent conversion over regenerated catalysts. The regenerated cycles have comparable conversion to fresh catalysts before any regeneration.

FIG. 7 shows oligomer selectivity over time for three cycles. Again, the first cycle is with fresh MTT zeolite catalyst and nickel on amorphous silica alumina catalyst arranged in a stacked bed configuration in the pilot plant reactor indicated by the dashes. The second cycle in triangles and the third cycle in circles represent conversion over regenerated catalysts. The regenerated cycles have comparable selectivities to fresh catalysts before any regeneration.

### Specific Embodiments

While the following is described in conjunction with specific embodiments, it will be understood that this description is intended to illustrate and not limit the scope of the preceding description and the appended claims.

A first embodiment of the invention is a process for oligomerizing an olefin stream comprising oligomerizing a charge olefin stream over an oligomerization catalyst bed to produce an oligomerized olefin stream; purging the oligomerization catalyst bed with an inert gas stream to remove hydrocarbons from the oligomerization catalyst bed; and regenerating the oligomerization catalyst bed by contact with an oxygen supply gas. An embodiment of the invention is one, any or all of prior embodiments in this paragraph up through the first embodiment in this paragraph wherein the inert gas stream comprises carbon dioxide. An embodiment of the invention is one, any or all of prior embodiments in this paragraph up through the first embodiment in this paragraph further comprising fermenting starch and/or sugars to produce an alcohol stream and a carbon dioxide stream and providing the inert stream from the carbon dioxide stream. An embodiment of the invention is one, any or all of prior embodiments in this paragraph up through the first embodiment in this paragraph further comprising pressuring the oligomerization catalyst bed with the inert gas after regeneration. An embodiment of the invention is one, any or all of prior embodiments in this paragraph up through the first embodiment in this paragraph further comprising combusting carbon from the oligomerization catalyst to produce a carbon dioxide stream and providing the inert gas stream from the carbon dioxide stream. An embodiment of the invention is one, any or all of prior embodiments in this paragraph up through the first embodiment in this paragraph further comprising venting vapors from the oligomerization catalyst bed and draining hydrocarbon liquid from the oligomerization catalyst bed before purging. An embodiment of the invention is one, any or all of prior embodiments in this paragraph up through the first embodiment in this paragraph further comprising converting the alcohol stream to an ethylene stream and providing the charge olefin stream from the ethylene stream. An embodiment of the invention is one, any or all of prior embodiments in this paragraph up through the first embodiment in this paragraph wherein the oligomerization catalyst bed is a first stage oligomerization catalyst bed and further comprising oligomerizing the oligomerized olefin stream over a second stage oligomerization catalyst bed to produce an oligomerate stream. An embodiment of the invention is one, any or all of prior embodiments in this paragraph up through the first embodiment in this paragraph further comprising purging the second stage oligomerization catalyst bed with an inert gas stream to remove hydrocarbons from the second stage oligomerization catalyst bed; and regenerating the second stage oligomerization catalyst bed by contact with an oxygen supply gas. An embodiment of the invention is one, any or all of prior embodiments in this paragraph up through the first embodiment in this paragraph wherein the inert gas stream is carbon dioxide. An embodiment of the invention is one, any or all of prior embodiments in this paragraph up through the first embodiment in this paragraph further comprising fermenting starch and/or sugars to produce an alcohol stream and a carbon dioxide stream and providing the inert stream from the carbon dioxide stream.

A second embodiment of the invention is a process for oligomerizing an olefin stream comprising fermenting starch and/or sugars to produce alcohol and carbon dioxide and providing an inert, carbon dioxide stream; converting the alcohol to an ethylene stream; providing a charge olefin stream from the ethylene stream; oligomerizing a charge olefin stream over an oligomerization catalyst bed to produce an oligomerized olefin stream; purging the oligomerization catalyst bed with an inert, carbon dioxide stream to remove hydrocarbons from the oligomerization catalyst bed; and regenerating the oligomerization catalyst bed by contact with an oxygen supply gas. The process of claim 12 further comprising venting vapors from the oligomerization catalyst bed and draining hydrocarbon liquid from the oligomerization catalyst bed before purging. An embodiment of the invention is one, any or all of prior embodiments in this paragraph up through the second embodiment in this paragraph wherein the oligomerization catalyst bed is a first stage oligomerization catalyst bed and further comprising oligomerizing the oligomerized olefin stream over a second stage oligomerization catalyst bed to produce an oligomerate stream. An embodiment of the invention is one, any or all of prior embodiments in this paragraph up through the second embodiment in this paragraph further comprising pressuring the oligomerization catalyst bed with the inert, carbon dioxide stream after regeneration. An embodiment of the invention is one, any or all of prior embodiments in this paragraph up through the second embodiment in this paragraph further comprising filling the oligomerization catalyst bed with a heavy hydrocarbon after regeneration.

A third embodiment of the invention is a process for oligomerizing olefins comprising oligomerizing an olefin stream over a first stage oligomerization catalyst bed to produce a oligomerized olefin stream; purging the first stage oligomerization catalyst bed with an inert gas stream; regenerating the first stage oligomerization catalyst bed; oligomerizing the oligomerized olefin stream over a second stage oligomerization catalyst bed to produce an oligomerate stream; purging the second stage oligomerization catalyst bed with an inert gas stream; regenerating the second stage oligomerization catalyst bed. An embodiment of the invention is one, any or all of prior embodiments in this paragraph up through the third embodiment in this paragraph further comprising fermenting starch and/or sugars to produce an alcohol stream and a carbon dioxide stream and providing the inert gas stream from the carbon dioxide stream. An embodiment of the invention is one, any or all of prior embodiments in this paragraph up through the third embodiment in this paragraph further comprising fermenting starch and/or sugars to produce an alcohol stream and a carbon dioxide stream and providing the olefin stream from the alcohol stream. An embodiment of the invention is one, any or all of prior embodiments in this paragraph up through the third embodiment in this paragraph further comprising pressuring the oligomerization catalyst bed with the inert gas after regeneration.

Without further elaboration, it is believed that using the preceding description that one skilled in the art can utilize the present disclosure to its fullest extent and easily ascertain the essential characteristics of this disclosure, without departing from the spirit and scope thereof, to make various changes and modifications of the disclosure and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process for oligomerizing an olefin stream comprising:
oligomerizing a charge olefin stream over an oligomerization catalyst bed to produce an oligomerized olefin stream;
purging said oligomerization catalyst bed with an inert gas stream to remove hydrocarbons from the oligomerization catalyst bed; and
regenerating said oligomerization catalyst bed by contact with an oxygen supply gas.

2. The process of claim 1 wherein said inert gas stream comprises carbon dioxide.

3. The process of claim 1 further comprising fermenting starch and/or sugars to produce an alcohol stream and a carbon dioxide stream and providing said inert stream from said carbon dioxide stream.

4. The process of claim 2 further comprising pressurizing said oligomerization catalyst bed with said inert gas after regeneration.

5. The process of claim 1 further comprising combusting carbon from said oligomerization catalyst to produce a carbon dioxide stream and providing said inert gas stream from said carbon dioxide stream.

6. The process of claim 5 further comprising venting vapors from said oligomerization catalyst bed and draining hydrocarbon liquid from said oligomerization catalyst bed before purging.

7. The process of claim 3 further comprising converting said alcohol stream to an ethylene stream and providing said charge olefin stream from said ethylene stream.

8. The process of claim 1 wherein said oligomerization catalyst bed is a first stage oligomerization catalyst bed and further comprising oligomerizing said oligomerized olefin stream over a second stage oligomerization catalyst bed to produce an oligomerate stream.

9. The process of claim 8 further comprising purging said second stage oligomerization catalyst bed with an inert gas stream to remove hydrocarbons from the second stage oligomerization catalyst bed; and
regenerating said second stage oligomerization catalyst bed by contact with an oxygen supply gas.

10. The process of claim 9 wherein said inert gas stream is carbon dioxide.

11. The process of claim 10 further comprising fermenting starch and/or sugars to produce an alcohol stream and a carbon dioxide stream and providing said inert stream from said carbon dioxide stream.

12. A process for oligomerizing an olefin stream comprising:
fermenting starch and/or sugars to produce alcohol and carbon dioxide and providing an inert, carbon dioxide stream;
converting said alcohol to an ethylene stream;
providing a charge olefin stream from said ethylene stream;
oligomerizing a charge olefin stream over an oligomerization catalyst bed to produce an oligomerized olefin stream;
purging said oligomerization catalyst bed with an inert, carbon dioxide stream to remove hydrocarbons from the oligomerization catalyst bed; and
regenerating said oligomerization catalyst bed by contact with an oxygen supply gas.

13. The process of claim 12 further comprising venting vapors from said oligomerization catalyst bed and draining hydrocarbon liquid from said oligomerization catalyst bed before purging.

14. The process of claim 12 wherein said oligomerization catalyst bed is a first stage oligomerization catalyst bed and further comprising oligomerizing said oligomerized olefin stream over a second stage oligomerization catalyst bed to produce an oligomerate stream.

15. The process of claim 12 further comprising pressuring said oligomerization catalyst bed with said inert, carbon dioxide stream after regeneration.

16. The process of claim 12 further comprising filling said oligomerization catalyst bed with a hydrocarbon after regeneration.

17. A process for oligomerizing olefins comprising:
oligomerizing an olefin stream over a first stage oligomerization catalyst bed to produce an oligomerized olefin stream;
purging said first stage oligomerization catalyst bed with an inert gas stream;
regenerating said first stage oligomerization catalyst bed;
oligomerizing said oligomerized olefin stream over a second stage oligomerization catalyst bed to produce an oligomerate stream;
purging said second stage oligomerization catalyst bed with an inert gas stream;
regenerating said second stage oligomerization catalyst bed.

18. The process of claim 15 further comprising fermenting starch and/or sugars to produce an alcohol stream and a carbon dioxide stream and providing said inert gas stream from said carbon dioxide stream.

19. The process of claim 15 further comprising fermenting starch and/or sugars to produce an alcohol stream and a carbon dioxide stream and providing said olefin stream from said alcohol stream.

20. The process of claim 17 further comprising pressuring said oligomerization catalyst bed with said inert gas after regeneration.
